(19) United States
(12) Patent Application Publication
(10) Pub. No.: US 2006/0045797 A1
(43) Pub. Date: Mar. 2, 2006

(54) System for treating bio-hazardous medical waste

(76) Inventor: Don Frazier, Portland, OR (US)

Correspondence Address: KLARQUIST SPARKMAN, LLP, 121 SW SALMON STREET, SUITE 1600, PORTLAND, OR, 97204, US

(21) Appl. No.: 10/928,043

(22) Filed: Aug. 27, 2004

(51) Int. Cl. A61L 2/00; A62B 7/08

(52) U.S. Cl. 422/22; 422/122

(57) ABSTRACT

The present disclosure concerns embodiments of a system for rendering bio-hazardous medical waste safe for disposal. In accordance with one embodiment, a method for treating medical waste material comprises sterilizing the waste material, fractionating the waste material, and liquefying at least a portion of the waste material so as to form a mixture or slurry of liquefied waste material and solid, fractionated waste material. The slurry is then solidified to form a sterile, unitary mass that occupies a volume that is less than the volume of the waste material prior to being treated.

### FIELD

This application relates to a system for treating bio-hazardous medical waste, and more particularly to sterilizing and densifying medical waste for disposal.

### BACKGROUND

The safe handling and disposal of regulated medical waste from various medical and health-care facilities presents multiple concerns. Foremost, medical waste represents a biological hazard. Accordingly, medical waste typically must be sterilized through heat, chemical, or other methods prior to its subsequent handling and disposal. Another problem concerns the safe handling of so called “sharps” waste, which typically includes contaminated needles, scalpels, razors, lances, and other sharp metal or glass objects. Even after such items are sterilized, they remain a physical risk to waste handlers and create additional problems in waste packaging.

An additional problem relates to the sheer volume of medical waste generated by hospitals and other health-care facilities. About one million tons of medical waste is generated each year in the United States. Thus, waste-generating facilities must incur substantial expense to have a licensed waste hauler collect and dispose of medical wastes. On-site treatment systems are an alternative to the collection and disposal of waste by a licensed hauler. However, such systems have not proven to be practical or cost-efficient.

Finally, traditional methods of handling medical waste have done very little to alter the form, structure, and physical identity of the waste. Recognizable medical waste is an obstacle to introducing medical waste into the normal sanitation waste stream. As a result, there is a need to alter the physical state of the medical waste so as to produce an unrecognizable, yet harmless waste product. In addition, there is a strong need to physically reduce the overall volume of solid waste prior to disposal, especially in areas where solid waste is disposed of through burial in landfills.

Accordingly, there remains much room for improvement and variation within the art.

### SUMMARY

According to one aspect, described below are embodiments that provide an on-site, medical waste treatment apparatus that sterilizes, fractionates, and densifies medical waste, thereby reducing the overall volume of the waste and rendering the waste unrecognizable and safe for disposal. In certain embodiments, the treatment apparatus is used to process used medical items, such as needles, scalpels, razors, lances, and other sharp metal or glass objects, that are contained in plastic sharps containers. The treatment apparatus can be installed in any convenient location inside a hospital or other health-care facility where such sharps containers are used.

In use, an operator loads sharps containers filled with used medical items into the treatment apparatus. The treatment apparatus automatically fractionates each container and its contents, and heats the waste to a temperature sufficient to sterilize the waste and melt the thermoplastic components of the waste to form a slurry of thermoplastic resin and solid waste items. As used herein, “fractionating” waste material means to divide, break up, shred, slice, cut, or otherwise separate the waste material into smaller pieces.

The slurry is allowed to solidify and form a sterile, unitary mass of fractionated waste items encapsulated within the thermoplastic resin. The sterile mass is biologically safe and can be handled by personnel as conventional waste without the need for additional protective measures.

In one representative embodiment, an apparatus for treating infectious medical waste material includes a processing chamber adapted to receive the waste material (e.g., waste items in a sharps container). A fractionation device is disposed in the processing chamber. As the waste material passes through the fractionation device, the waste material is fractionated into smaller, unrecognizable pieces. A heating source is configured to heat the waste material at a temperature sufficient to sterilize the waste material and melt the thermoplastic components of the waste material so as to form a mixture of molten and solid waste material. A cooling chamber receives the mixture of molten and solid waste material, which solidifies to form a unitary, sterile mass for disposal.

In another representative embodiment, a method for treating medical waste material comprises sterilizing the waste material, fractionating the waste material, and liquefying at least a portion of the medical waste material so as to form a mixture or slurry of liquefied waste material and solid, fractionated waste material. The slurry is then solidified to form a sterile, unitary mass that occupies a volume that is less than the volume of the waste material prior to being treated.

The foregoing and other features and advantages of the invention will become more apparent from the following detailed description of several embodiments, which proceeds with reference to the accompanying figures.

### BRIEF DESCRIPTION OF THE DRAWINGS

FIG. 1 is a block diagram of a system for treating infectious medical waste, according to one embodiment.

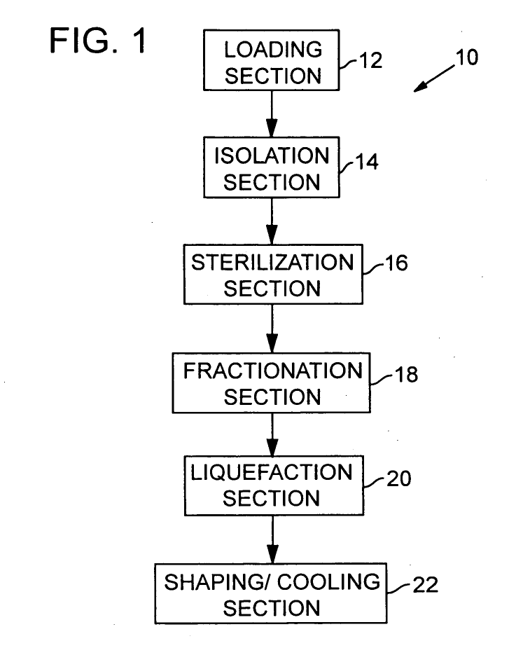

FIG. 2 is a schematic illustration of an on-site apparatus for treating infectious medical waste, according to one embodiment.

FIG. 3 is a vertical cross-sectional view of the apparatus of FIG. 2.

### DETAILED DESCRIPTION

As used herein, the singular forms “a,” “an,” and “the” refer to one or more than one, unless the context clearly dictates otherwise.

As used herein, the term “includes” means “comprises.”

As used herein, a group of individual members stated in the alternative includes embodiments relating to a single member of the group or combinations of multiple members. For example, the term “a, b, c,” includes embodiments relating to “a,” “b,” “a and b,” “a and c,” “b and c,” and “a, b, and c.”

Referring first to FIG. I, there is shown a block diagram of one embodiment of a system 10 that is used to render infectious medical waste safe for disposal. The illustrated system 10 generally includes a loading section 12, an isolation section 14, a sterilization section 16, a fractionation section 18, a liquefaction section 20, and a shaping/cooling section 22. In particular embodiments, the system 10 is implemented as a relatively compact unit that can be located inside a waste-generating facility where it can be easily accessed by personnel.

The loading section 12 receives the medical waste to be treated. Typically, the medical waste is contained in a plastic enclosure, or container, such as a conventional sharps container. In such cases, the enclosure itself is processed by the system along with its contents. A conveyor or similar device can be implemented to automatically transfer a container of medical waste from the loading section to the isolation section 14.

The isolation section 14 may comprise a fluid-tight chamber or similar structure that is dimensioned to receive a container of medical waste. Once the container is inside the isolation chamber, a vacuum source is activated to remove substantially all air from the chamber. The purpose of drawing a vacuum on the isolation chamber is to minimize the presence of oxygen in the system that could lead to combustion during subsequent processing of the waste. In lieu of or in addition to using vacuum to remove air from the isolation chamber, an inert gas (e.g., nitrogen or argon) can be used to purge the air from the isolation chamber.

The isolation section 14 also can be used as a pre-heating section in which the container of medical waste is heated from room temperature to a prescribed temperature prior to sterilization. Any of various heating methods and techniques can be used to pre-heat the medical waste. For example, waste heat from any of the subsequent sections of the system can be reclaimed to pre-heat the medical waste in the isolation section 14.

Following the isolation section 14, the medical waste enters a sterilization section 16, a fractionation section 18, and a liquefaction section 20, although not necessarily in this particular order. In certain embodiments, a single processing chamber contains the sterilization section, the fractionation section, and the liquefaction section. In other embodiments, the sterilization section 16, the fractionation section 18, and the liquefaction section 20 can comprise respective processing chambers. Nonetheless, in the sterilization section 16, the waste is heated to a prescribed temperature at which sterilization can occur. Sterilization typically occurs at temperatures of 110° C. or greater. Heating of the waste can be accomplished using any of various conventional heating techniques, including, but not limited to, microwave heating, radiant heating, atmospheric plasma heating, or steam heating.

In the fractionation section 18, the medical waste is fractionated into smaller pieces, thereby rendering the waste unrecognizable. As used herein, “fractionating” waste material means to divide, break up, shred, slice, cut, or otherwise separate the waste material into smaller pieces. Any of various devices can be used to fractionate the medical waste. For example, the medical waste can be passed through a series of rotating blades that function to shred the waste. In another example, the waste can be passed through a series of heated knives or cutting blades. Fractionation of the waste can occur prior to, after, or concurrently with the sterilization process.

In the liquefaction section 20, the waste is heated to a temperature sufficient to melt any thermoplastic components of the waste material (e.g., the sharps container and the plastic portions of needles) so as to form a slurry or mixture of liquefied thermoplastic resin and solid, fractionated waste items. If the sterilization process and the liquefaction process take place in a single processing chamber, the heating source used to sterilize the waste also can be used to liquefy the thermoplastic components of the waste.

The mixture of liquefied and solid waste is transferred to the shaping/cooling section 22, in which the mixture is allowed to solidify into a generally sterile, unitary mass that can be introduced into a conventional waste stream. Because the thermoplastic material encapsulates the solid waste items, including any dangerous sharps materials, the mass is rendered physically safe for handling. The shaping/cooling section 22 can be a separate chamber that functions as a mold for shaping the unitary mass. The slurry can be exposed to a cooling medium (e.g., a flow of cold water or gas, such as air) to facilitate the solidification process and bring the temperature of the waste down to a reduced temperature (e.g., room temperature) for further processing or handling.

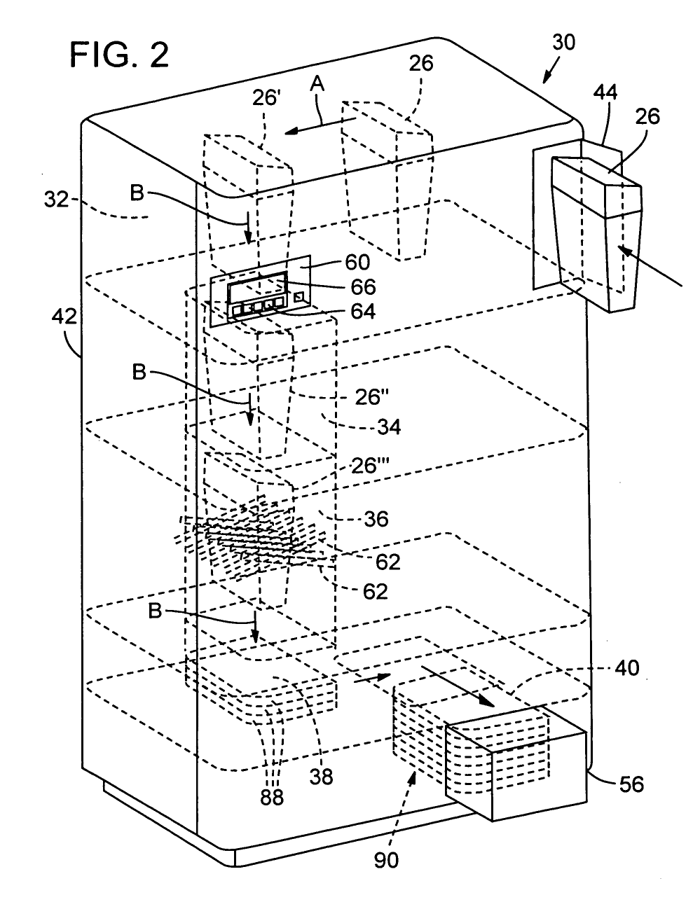

FIG. 2 is a schematic illustration of an apparatus 30 for treating infectious medical waste, according to one embodiment. The apparatus 30 in the illustrated embodiment generally includes a loading stage 32, an isolation chamber 34, a processing chamber 36, a solidification chamber 38 (also referred to herein as a cooling chamber), and a storage section 40, all of which are housed within a housing or enclosure 42. The apparatus 30 can be used as an on-site treatment device for conveniently treating medical waste at its point of origin. The housing 42 desirably is of a size that permits the apparatus to be transported through standard-sized doorways in hospitals and other health-care facilities. Thus, the treatment apparatus 30 can be installed in any convenient location in a health-care facility where medical waste is generated. For example, in a large hospital, it would be desirable to install a treatment apparatus on each floor of the hospital. In this manner, medical waste can be quickly and efficiently treated at its point of origin. In certain embodiments, the treatment apparatus 30 is powered by a standard 110 VAC power supply, although other power sources also can be used.

The apparatus 30 is depicted as being used to process medical waste items contained in standard sharps containers 26. Thus, the following description of the apparatus 30 proceeds with reference to processing medical waste contained in containers 26. However, in other applications, the apparatus 30 can be used to process waste items contained in so-called “red bags” or other soft or flexible containers. Alternatively, the apparatus 30 also can be used to process loose waste items that are not contained in an enclosure.

As shown in FIG. 2, the apparatus 30 also can include a controller 60 (also referred to herein as a control unit) that can be programmed to control the operation of various components of the apparatus and to monitor and record various operating parameters of the apparatus. In particular embodiments, the operation of the apparatus 30 is completely automated and under the control of the controller 60.

The controller 60 may be linked to communicate with an external computing device (not shown), which can be a microcomputer or a general purpose desktop or laptop computer. Any of various linkage devices or techniques may be used to communicate between the controller 60 and the external computing device, such as an infrared emitter, radio waves, modems, Bluetooth® wireless technology, 802.11 wireless technology, direct connections, and the like. The controller also may be equipped to receive a removable data-storage device, such as a removable memory card (e.g., a flash memory card).

The controller 60 may include an input keypad 64, which can be an alpha or alpha numeric keypad. Using keypad 64, a user may input different operating parameters of the apparatus. The controller also may include a display screen 66, which can be a liquid crystal display (LCD), to indicate which selections have been made using keypad 64 and/or to display different operating parameters of the apparatus.

The apparatus 30 includes a door 44 that provides access to the loading stage 32 for loading containers 26 into the apparatus for processing. As shown in FIG. 2, the loading stage 32 desirably is sized to hold multiple containers 26 that are to be processed. For safety purposes, the door 44 is configured to prevent removal of a container 26 from the loading stage except by authorized maintenance personnel. Prior to being loaded into the loading stage 32, each container 26 can be logged for regulatory traceability, such as by scanning a barcode on the container or manually entering into the controller 60 the serial number or other identification number of the container.

A conveyor or similar mechanism (not shown) can be implemented in the loading stage 32 to automatically move the containers 26 through the loading stage in the direction of arrow A to position above the isolation chamber 34, as depicted by container 26′. Container 26′ is then gravity fed through the isolation chamber 34, the processing chamber 36, and the solidification chamber 38 in the direction of arrow B for processing.

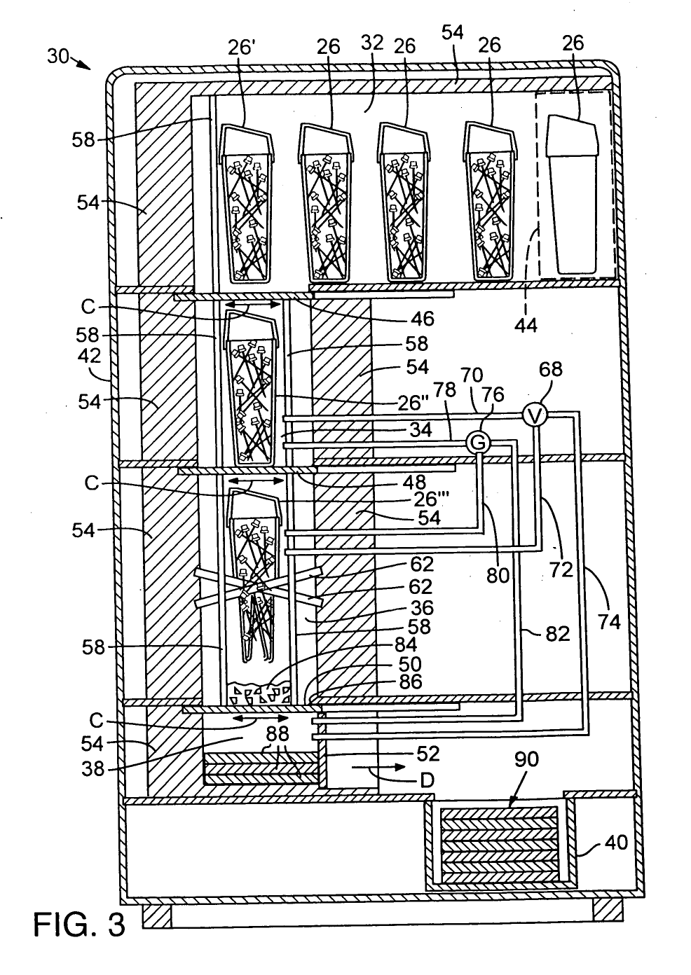

As shown in FIG. 3, a first gate valve 46 is disposed between the loading stage 32 and the isolation chamber 34; a second gate valve 48 is disposed between the isolation chamber 34 and the processing chamber 36; and a third gate valve 50 is disposed between the processing chamber 36 and the solidification chamber 38. Each of the gate valves 46, 48, and 50 is movable in a lateral direction, as indicated by double-headed arrows C, between respective open and closed positions to control the movement of waste items through the chambers 34, 36, and 38. FIG. 3 shows each gate valve 46, 48, and 50 in a closed position. Moving a gate valve laterally away from its closed position (to the right, as viewed in FIG. 3) opens a path between adjacent sections of the apparatus through which waste material can pass. Movement of the gate valves 46, 48, and 50 can be accomplished using conventional techniques. For example, the gate valves can be electronically or pneumatically actuated and under the control of the controller 60 (FIG. 2).

The gate valves 46, 48, and 58 desirably are configured to provide a fluid-tight seal across their respective openings when they are closed. This prevents gases from escaping the isolation chamber 34, the processing chamber 36, and the solidification chamber 38 as waste items are processed in these chambers.

Mechanisms other than the illustrated gate valves can be employed to control the movement of waste material through the apparatus and/or to seal each chamber during processing. For example, a hinged door or similar structure can be used in place of each gate valve 46, 48, and 50.

To feed container 26′ into the isolation chamber 34, the second gate valve 48 is closed, as shown in FIG. 3, and the first gate valve 46 is opened, which allows container 26′ to drop into the isolation chamber where it is temporally supported on top of the second gate valve 48, as depicted by container 26″. When processing inside the isolation chamber 34 is complete, the second gate valve 48 is opened, thereby allowing container 26″ to fall by gravity into the processing chamber 38, as depicted by container 26′″. When the second gate valve 48 is opened to allow container 26″ to drop into the processing chamber, the first gate valve 46 remains closed to prevent any gases from the isolation chamber 34 or the processing chamber 36 from escaping to the loading stage 32. After container 26′″ is deposited in the processing chamber, the second gate valve 48 is closed, and the first gate valve 46 is opened to allow the next container in the loading stage 32 to drop into the isolation chamber 34.

Inside the processing chamber 36, the container 26′″ and its contents (e.g., needles, scalpels, etc.) are sterilized, fractionated, and at least partially liquefied, as described in greater detail below. When processing inside the processing chamber 36 is complete, the third gate valve 50 is opened to allow the processed waste material, which is a mixture of liquefied waste and solid, fractionated waste items, to fall by gravity into the solidification chamber 38.

As shown in FIG. 3, the apparatus 30 may include guide rails 58 for guiding the containers 26 as they move vertically through the apparatus. Insulation 54 substantially encloses the isolation chamber 34 and the processing chamber 36 to minimize heat loss to the surrounding environment. The insulation 54 can be made from any of various materials known in the art. Insulation 54 also can be used to partially or completely enclose the loading stage 32 and the solidification chamber 38.

The apparatus 30 in the illustrated embodiment includes a vacuum source 68 that is fluidly connectable to the isolation chamber 34, the processing chamber 36, and the solidification chamber 38 via vacuum lines 70, 72, and 74, respectively. The vacuum source 68 can be small vacuum pump mounted inside the apparatus. In alternative embodiments, the vacuum source can be a centralized house vacuum system, as typically found in hospitals and other health-care facilities. In addition, a source 76 of a pressurized, inert gas (e.g., argon or nitrogen) desirably is fluidly connectable to the isolation chamber 34, the processing chamber 36, and the solidification chamber 38 via gas lines 78, 80, and 82, respectively. Control of vacuum or pressurized gas to the chambers 34, 36, and 38 can be accomplished by control valves (not shown) in the vacuum and gas lines. Gases displaced from the interiors of the chambers 34, 36, and 38 can be filtered through a suitable filter (e.g., an activated carbon filter) mounted inside the apparatus 30 or at a remote location.

The isolation chamber 34 functions to establish a substantially oxygen-free atmosphere for processing the waste material. When container 26″ is loaded into the isolation chamber 34, the first gate valve 46 is closed to provide a substantially fluid-tight chamber. Thereafter, the vacuum from the vacuum source 68 is controlled to evacuate the atmosphere inside the isolation chamber. Removal of substantially all oxygen from the isolation chamber 34 avoids combustion of the waste material in the processing chamber 36.

The inert gas from the inert-gas source 76 can be used in lieu of or in addition to the vacuum source 68 for removing the air from the isolation chamber 34. In one implementation, for example, the isolation chamber 34 is initially purged with the inert gas and the atmosphere inside the chamber is subsequently evacuated by activating the vacuum source 68. In another implementation, a vacuum source is not provided, and the inert gas is used to displace the air inside the isolation chamber. In the latter implementation, the displaced air is forced to flow through an exhaust line (not shown) connected to the isolation chamber.

The isolation chamber 34 also can have a heating source to pre-heat container 26″ and its contents from room temperature to a prescribed temperature prior to downstream processing in the processing chamber 36. In certain embodiments, the waste material in the isolation chamber is heated approximately to the temperature at which the waste material will be subsequently sterilized in the processing chamber (e.g., 250° C.-300° C.). This decreases the residence time required for sterilization in the processing chamber and increases the total throughput of the apparatus.

The heating source for the isolation chamber 36 can be heated gas from the gas source 76. To minimize energy consumed by the apparatus, the gas from the gas source 76 can be heated using waste heat rejected from the processing chamber 36. Other heating sources also can be used. For example, an electric heating element can be disposed in the isolation chamber for pre-heating the waste material. In another example, microwaves from a microwave source are introduced into the isolation chamber for pre-heating the waste material.

The processing chamber 36 in the illustrated configuration includes a heating source (not shown) for sterilizing and liquefying (i.e., melting) the thermoplastic components of the waste material (including container 26′″), and a fractionation device for fractionating (i.e., breaking up) the waste material into smaller pieces. Consequently, sterilization, liquefaction and fractionation occur generally concurrently with each other in the processing chamber. In other embodiments, sterilization, fractionation, and liquefaction of the waste material can be accomplished in separate chambers or sections of the apparatus. Further, one or more of the sterilization, the fractionation, and the liquefaction processes can be staged over more than one chamber or section of the apparatus. For example, the waste material can be passed through plural fractionation stages connected in series.

The heating source used to heat the waste material in the processing chamber 36 can be, for example, a microwave heating source, a radiant heater, or a plasma heating device, as known in the art. The heating source heats the waste material at a temperature sufficient to sterilize the waste material and to cause the thermoplastic components of the waste material to soften or completely liquefy. Typically, a minimum temperature of about 160° C. is required to effectively sterilize the waste and liquefy most thermoplastic waste items. Desirably, the temperature inside the processing chamber is maintained below the combustion and pyrolysis temperatures of the waste items being processed. In certain embodiments, for example, the waste material is heated within the temperature range of about 160° C. to about 350° C., and more preferably within the temperature range of about 250° C. and about 350° C. As the waste material is heated, the vacuum source 68 maintains a vacuum inside the processing chamber to avoid pressurizing the processing chamber 36, thereby further reducing the risk of possible combustion and pyrolysis. If desired, the inert. gas from the gas source 76 can be introduced into the processing chamber via the gas line 80 as additional protection against combustion and pyrolysis.

In the illustrated embodiment, the fractionation device is depicted as an array of heated cutting blades or knives 62. As the container 26′″ passes through the knives 62, the knives 62 cut through the container 26′″ and the waste items inside the container, causing these items to break up into smaller pieces. Fractionating the waste material serves three main purposes. First, fractionation densifies of the waste material. Second, sterilization is facilitated through fractionation since more surface area is exposed to the heating source. Third, fractionation renders the waste material unrecognizable.

As best shown in FIG. 2, the knives 62 are supported at an angle with respect to the vertical side walls of the processing chamber 36, with each knife 62 extending downwardly and inwardly but in an opposite direction from an adjacent knife 62. This configuration directs the waste material away from the side walls of the processing chamber. The knives 62 can be operated to vibrate or move laterally toward and away from the center of the processing chamber to facilitate the flow of waste items through the knives 62. In addition, a ram or similar device can be implemented to physically push the container 26′″ through the knives 62 to facilitate the fractionation process.

As the waste material falls through the processing chamber 36, the heat applied to sterilize the waste material softens or completely melts all or most of the thermoplastic components. Other non-thermoplastic materials, including any metals, fabrics, paper, epoxies, and thermoset plastics, fall to the bottom of the processing chamber and form a mixture or slurry 84 of liquefied thermoplastic resin and solid waste items on top of the third gate valve 50. As the slurry 84 is formed, nearly all air volume is removed from the waste material, thereby resulting in further densification of the waste material.

When all of the waste material settles at the bottom of the processing chamber, either in liquid or solid form, the atmosphere inside the processing chamber 36 is vented into the solidification chamber 38 to equalize the temperature and pressure in the processing chamber and the solidification chamber. Venting of the atmosphere of the processing chamber to the solidification chamber can be accomplished by opening a valve in an equalization line (not shown) extending between the two chambers. Operation of the valve can be controlled by the controller 60. When equalization is established, the controller 60 opens the third gate valve 50, which allows the slurry 84 to pour into the solidification chamber 38. A scrapper blade 86 (FIG. 3) can be positioned at the bottom of the processing chamber to scrap away any material adhering to the upper surface of the third gate valve 50 as it is opened. Thereafter, the third gate valve 50 is closed and the second gate valve 48 is opened to allow the next container 26″ in the isolation chamber 34 to drop into the processing chamber 36.

Once in the solidification chamber 38, the slurry 84 is allowed to cool. This causes the thermoplastic resin to solidify as a unitary, sterile slug or puck 88, in which the solid, non-thermoplastic waste items are encapsulated by the thermoplastic resin. As a result of the fractionation and liquefaction processes, the solidified slug 88 occupies a much smaller volume than the overall volume of the container 26 from which the slug was formed.

As best shown in FIG. 3, the solidification chamber 38 desirably is dimensioned such that multiple slugs 88 can be formed on top of each other inside the chamber to form a brick 90. As a slug 88 solidifies, it adheres to a previously formed slug 88 so that the resulting brick 90 is a solid, unitary mass of material. To facilitate the solidification process, the internal surfaces of the solidification chamber can be cooled and/or cold purge gas from the gas source 76 can be introduced into the solidification chamber.

The internal surfaces of the solidification chamber serve as a mold for shaping each slug 88. The slugs desirably are shaped so as to have a uniform, relatively flat cross-sectional profile so as to maximize surface area for optimum cooling. In particular embodiments, for example, the slugs 88 have a thickness between about ¼ and ½ inch.

When the maximum number of slugs 88 are formed (e.g., six in the illustrated embodiment), a door 52 of the solidification chamber 38 is opened and the brick 90 of slugs 88 is ejected to the holding section 40. An ejection mechanism (not shown), such as a movable ram, automatically pushes the brick 90 through the opening in the solidification chamber, as indicated by arrow D, until the brick drops into the holding section. The holding section 40 desirably is sized to hold multiple bricks 90. After a brick is ejected from the solidification chamber, the door 52 is closed and the third gate valve 50 can be opened to receive a slurry 84 for forming the first slug 88 of a new brick.

When a predetermined number of bricks 90 are formed, the controller 60 alerts the user to remove the bricks from the apparatus 30. The holding section 40 can have a removable door 56 (FIG. 2) to permit user access to the holding section. Since the bricks are sterile, they can be safely handled by personnel and processed as ordinary waste. For example, the bricks can be incinerated or buried in a land fill. Alternatively, the bricks can be set aside for secondary processing. For example, the bricks can be sent to a processing facility to reclaim the metal in the bricks.

The present disclosure has been shown in the described embodiments for illustrative purposes only. The present disclosure may be subject to many modifications and changes without departing from the spirit or essential characteristics thereof. I therefore claim as my invention all such modifications as come within the spirit and scope of the following claims.

I claim:

1. A method for treating infectious medical waste material, the method comprising:
sterilizing the waste material;
fractionating at least a portion of the waste material;
liquefying at least a portion of the waste material to form a mixture of liquefied waste material and solid, fractionated waste material; and
solidifying the liquefied portion of the mixture so as to form a sterile, solid mass having an overall volume that is less than the volume of the waste material prior to being treated.

2. The method of claim 1, wherein the act of sterilizing the waste material and the act of fractionating the waste material occur generally concurrently.

3. The method of claim 1, wherein the temperature of the waste material during the acts of sterilizing, fractionating, and liquefying is maintained below the combustion temperature of the waste material.

4. The method of claim 1, wherein:
the waste material comprises thermoplastic waste material and non-thermoplastic waste material; and
liquefying at least a portion of the waste material comprises heating the waste material to a temperature sufficient to cause the thermoplastic waste material to melt and form a mixture of liquefied thermoplastic material and solid, non-thermoplastic waste material.

5. The method of claim 4, wherein the unitary mass comprises fractionated, non-thermoplastic waste material substantially encapsulated within solidified thermoplastic waste material.

6. The method of claim 1, wherein the act of solidifying the liquefied portion of the mixture comprises cooling the mixture.

7. The method of claim 1, wherein sterilizing the waste material comprises heating the waste material at about 250° C. to about 350° C. for a predetermined period of time.

8. The method of claim 1, wherein fractionating the waste material comprises shredding the waste material.

9. The method of claim 1, wherein the acts of sterilizing, fractionating, and liquefying are performed concurrently.

10. The method of claim 1, wherein the waste material is sterilized and liquefied in a substantially oxygen-free atmosphere.

11. The method of claim 10, wherein the waste material is fractionated in a substantially oxygen-free atmosphere.

12. The method of claim 1, wherein the waste material is sterilized, fractionated, and liquefied in a processing chamber.

13. The method of claim 12, further comprising purging the processing chamber with an inert gas prior to sterilizing, fractionating, and liquefying the waste material.

14. The method of claim 12, further comprising maintaining a vacuum in the processing chamber as the waste material is sterilized, fractionated, and liquefied.

15. The method of claim 12, wherein the waste material is gravity fed through the processing chamber.

16. The method of claim 1, wherein the waste material to be treated is contained in a sharps container which is sterilized, fractionated, liquefied, and solidified along with the waste material.

17. The method of claim 1, further comprising recovering metal from the unitary mass.

18. An apparatus for treating infectious medical waste material, at least a portion of which comprises thermoplastic material, the apparatus comprising:
a processing chamber adapted to receive the waste material;
a fractionation device disposed in the processing chamber and configured to separate at least a portion of the waste material into smaller pieces;
a heating source configured to heat the waste material at a temperature sufficient to sterilize the waste material and liquefy the thermoplastic material so as to form a mixture of molten and solid waste material; and
a cooling chamber in which the mixture of molten and solid waste material is cooled to form a unitary, sterile, and solidified mass for disposal.

19. The apparatus of claim 18, further comprising an isolation chamber in communication with the processing chamber.

20. The apparatus of claim 18, wherein the heating source is a microwave heating source configured to introduce microwave energy into the processing chamber to heat the waste material.

21. The apparatus of claim 18, wherein the temperature at which the waste material is heated by the heating source is lower than the combustion temperature of the waste material.

22. The apparatus of claim 21, wherein the fractionation device comprises a plurality of heated cutting blades configured to cut at least a portion of the waste material into smaller pieces as the waste material passes through the blades.

23. The apparatus of claim 22, wherein the cutting blades are configured to move relative to each other to facilitate cutting of the waste material.

24. The apparatus of claim 18, wherein fractionation device comprises a plurality of rotating shredding blades configured to shred the waste material.

25. The apparatus of claim 18, wherein:
the processing chamber has an inlet through which the waste material is introduced into the chamber and an outlet through which the mixture of molten and solid waste material exits the chamber; and
the apparatus further comprises first and second movable gate valves, the first gate valve being selectively operable to open and close the inlet of the processing chamber, the second gate valve being selectively operable to open and close the outlet of the processing chamber.

26. The apparatus of claim 18, further comprising a vacuum source that is fluidly connectable to the processing chamber for maintaining a vacuum in the processing chamber.

27. The apparatus of claim 18, wherein the cooling chamber serves as a mold that receives the mixture of molten and solid waste material and shapes the solidified mass from the mixture.

28. An apparatus for treating infectious medical waste material, at least a portion of which comprises thermoplastic material, the apparatus comprising:
means for separating at least a portion of the waste material into smaller pieces;
means for heating the waste material at a temperature sufficient to sterilize all of the waste material and liquefy the thermoplastic material so as to form a mixture of molten and solid waste material; and
means for cooling the mixture of molten and solid waste material to form a unitary, sterile, and solidified mass for disposal.

29. The apparatus of claim 28, wherein the means for heating the waste material comprises a microwave heating source.

30. The apparatus of claim 28, wherein the means for breaking up the waste material into smaller pieces comprises a plurality of heated cutting blades configured to cut at least a portion of the waste material into smaller pieces as the waste material passes through the blades.

31. The apparatus of claim 28, further comprising:
a processing chamber in which the waste material is fractionated and sterilized, and the thermoplastic material is liquefied to form a mixture of molten and solid waste material; and
a cooling chamber that receives the mixture of molten and solid waste material from the processing chamber.

32. A method for treating medical waste material, the method comprising:
separating at least a portion of the waste material into smaller pieces;
heating the waste material at a temperature sufficient to kill microorganisms on the material and to begin melting at least some of the material; and
cooling the material to form a generally solid mass for disposal.

33. A method for treating medical waste material, the method comprising:
heating the waste material at a temperature sufficient to kill microorganisms on the material and to begin melting at least some of the material; and
cooling the material in a cooling chamber so as to form a generally solid mass for disposal by flowing a fluid over the material, wherein the temperature of the fluid is less than room temperature.

34. The method of claim 33, further comprising separating at least a portion of the waste material into smaller pieces prior to the act of cooling the material.

35. An apparatus for treating infectious medical waste material, the apparatus comprising:
at least one processing chamber adapted to receive the waste material;
a heating source configured to heat the waste material in the at least one processing chamber at a temperature sufficient to kill microorganisms on the waste material and melt at least some of the material, the resulting molten material mixing with any remaining other material; and
a cooling chamber that receives the mixture from the at least one processing chamber and in which the mixture is allowed to solidify for easy disposal.

36. The apparatus of claim 35, further comprising a fractionation device disposed in the at least one processing chamber and configured to separate at least a portion of the waste material into smaller pieces.
